(19) United States
(12) Patent Application Publication
(10) Pub. No.: US 2022/0359082 A1
(43) Pub. Date: Nov. 10, 2022

(54) HEALTH STATE PREDICTION SYSTEM INCLUDING ENSEMBLE PREDICTION MODEL AND OPERATION METHOD THEREOF

(71) Applicant: ELECTRONICS AND TELECOMMUNICATIONS RESEARCH INSTITUTE, Daejeon (KR)

(72) Inventors: Myung-eun LIM, Daejeon (KR); Do Hyeun KIM, Daejeon (KR); Jae Hun CHOI, Daejeon (KR)

(73) Assignee: ELECTRONICS AND TELECOMMUNICATIONS RESEARCH INSTITUTE, Daejeon (KR)

(21) Appl. No.: 17/735,320

(22) Filed: May 3, 2022

(30) Foreign Application Priority Data
May 4, 2021 (KR) 10-2021-0057828

(51) Int. Cl. G16H 50/30; G06N 20/20

(52) U.S. Cl. CPC G16H 50/30; G06N 20/20

(57) ABSTRACT

Disclosed is an operation method of a health state prediction system which includes an ensemble prediction model. The operation method includes sending a prediction result request for health time-series data to a plurality of external medical support systems, receiving a plurality of external prediction results associated with the health time-series data from the plurality of external medical support systems, generating long-term time-series data and short-term time-series data for each of the health time-series data, and the plurality of external prediction results, extracting a plurality of long-term trends based on the long-term time-series data, extracting a plurality of short-term trends based on the short-term time-series data, calculating external prediction goodness-of-fit based on the plurality of long-term trends and the plurality of short-term trends, and generating an ensemble prediction result based on the external prediction goodness-of-fit and the plurality of external prediction results.

### CROSS-REFERENCE TO RELATED APPLICATIONS

This application claims priority under 35 U.S.C. § 119 to Korean Patent Application No. 10-2021-0057828 filed on May 4, 2021, in the Korean Intellectual Property Office, the disclosures of which are incorporated by reference herein in their entireties.

### BACKGROUND

Embodiments of the present disclosure described herein relate to a technology for processing data, and more particularly, relate a health state prediction system including an ensemble prediction model and an operation method thereof.

To lead a healthy life, there is a demand for predicting the future health state in addition to treating current diseases. To predict the future health state, there is an increasing demand for diagnosing diseases or predicting future disease risk by analyzing big data. The development of industrial technologies and the information and communication technologies is supporting the construction of big data. In addition, technologies, which provide various services by training an electronic device (e.g., a computer) by using the big data, such as artificial intelligence are emerging. In particular, to predict the future health state, a way to build a prediction model using various medical data or health data is being proposed.

For accurate prediction, the larger the size of data, the more advantageous. However, it may be difficult to share data between various medical institutions due to various causes such as an ethical issue, a legal issue, and a personal privacy issue. In other words, it is difficult to construct one integrated medical-related data. To solve the issues unique to the medical data, there is being sought a way to train an individual prediction model built for each medical institution and predict the future health state of a patient based on a prediction result of each medical institution.

### SUMMARY

Embodiments of the present disclosure provide a health state prediction system including an ensemble prediction model predicting a future health state with high reliability so as to support clinical decision of a medical personnel, and an operation method thereof.

According to an embodiment, an operation method of a health state prediction system which includes an ensemble prediction model includes sending a prediction result request for health time-series data to a first external medical support system and a second external medical support system, receiving a first external prediction result associated with the health time-series data from the first external medical support system, receiving a second external prediction result associated with the health time-series data from the second external medical support system, generating long-term time-series data and short-term time-series data for each of the health time-series data, the first external prediction result, and the second external prediction result, extracting a first long-term trend and a second long-term trend based on the long-term time-series data, extracting a first short-term trend and a second short-term trend based on the short-term time-series data, calculating external prediction goodness-of-fit based on the first and second long-term trends and the first and second short-term trends, and generating an ensemble prediction result based on the external prediction goodness-of-fit and the first and second external prediction results.

In an embodiment, the method further includes calculating an error based on the calculated external prediction goodness-of-fit and a real external goodness-of-fit, and adjusting a parameter of the ensemble prediction model based on the error.

In an embodiment, the real external goodness-of-fit is generated based on an experimental value of a prediction time point, a first external experimental value corresponding to the prediction time point, and a second external prediction result corresponding to the prediction time point.

In an embodiment, the number of features included in the long-term time-series data is equal to the number of features included in the health time-series data, and the number of features included in the short-term time-series data is less than the number of features included in the health time-series data.

In an embodiment, the first and second short-term trends and the first and second long-term trends correspond to at least one of a moving trend feature, a variability trend feature, or a moving momentum trend feature.

In an embodiment, the moving trend feature includes a moving feature and a trend transition feature, and the moving momentum trend feature includes a slope feature and a variation feature.

In an embodiment, the moving trend feature indicates a gradual change trend of a value of the long-term time-series data or the short-term time-series data, the variability trend feature includes a magnitude, a pattern, and a period of variability of a value in the long-term time-series data or the short-term time-series data, and the moving momentum trend feature indicates a change direction including an increase and a decrease of the long-term time-series data or the short-term time-series data, and a strength for the change direction.

In an embodiment, the extracting of the first and second long-term trends based on the long-term time-series data includes extracting features belonging to a window time interval from the long-term time-series data to generate a long-tern feature window, generating the first long-term trend based on the long-term feature window, and generating the second long-term trend based on the long-term feature window.

In an embodiment, the calculating of the external prediction goodness-of-fit based on the first and second long-term trends and the first and second short-term trends includes generating a long-term goodness-of-fit vector based on the first and second long-term trends, generating a short-term goodness-of-fit vector based on the first and second short-term trends, and calculating the external prediction goodness-of-fit based on the long-term goodness-of-fit vector and the short-term goodness-of-fit vector.

In an embodiment, the generating of the long-term goodness-of-fit vector based on the first and second long-term trends includes generating a first long-term goodness-of-fit feature vector based on the health time-series data corresponding to a first time point, the first and second external prediction results corresponding to the first time point, and the first and second long-term trends corresponding to the first time point, and generating a second long-term goodness-of-fit feature vector based on the first long-term goodness-of-fit feature vector, the health time-series data corresponding to a second time point after the first time point, the first and second external prediction results corresponding to the second time point, and the first and second long-term trends corresponding to the second time point.

According to an embodiment, a health state prediction system includes a first medical support system including a first clinical decision support system and a first prediction system, a second medical support system including a second clinical decision support system and a second prediction system, and a third medical support system including a third clinical decision support system and a third prediction system. The first prediction system includes a predictor management device that is connected with the first clinical decision support system, receives an ensemble prediction request and health time-series data from the first clinical decision support system, and sends an ensemble prediction request to the first clinical decision support system, an ensemble prediction device that receives a prediction execution request from the predictor management device, sends an external prediction result request to a predictor interworking device in response to the prediction execution request, receives merged data from the predictor interworking device, to input the merged data to an ensemble prediction model, and receives the ensemble prediction result from the ensemble prediction model, the predictor interworking device that sends a prediction result request and the health time-series data to the second and third medical support systems in response to the external prediction result request, receives a first external prediction result from the second medical support system, receives a second external prediction result from the third medical support system, merges the health time-series data and the first and second external prediction results to generate the merged data, and an ensemble prediction model that receives the merged data from the ensemble prediction device and generates the ensemble prediction result.

In an embodiment, the health state prediction system further includes a time-series prediction device that receives a prediction execution request and the health time-series data from the predictor interworking device, inputs the health time-series data to a time-series prediction model, and receives the time-series prediction result from the time-series prediction model.

In an embodiment, the predictor interworking device merges the health time-series data, the first and second external prediction results, and the time-series prediction result to generate the merged data.

### BRIEF DESCRIPTION OF THE FIGURES

The above and other objects and features of the present disclosure will become apparent by describing in detail embodiments thereof with reference to the accompanying drawings.

FIG. 1 is a block diagram illustrating a health state prediction system according to an embodiment of the present disclosure.

FIG. 2 is a block diagram illustrating an example of a prediction system of FIG. 1.

FIG. 3 is a flowchart illustrating an example of an operation of a prediction system of FIG. 1.

FIG. 4 is a flowchart illustrating an example of an operation of a prediction system of FIG. 1.

FIG. 5 is a flowchart illustrating an example of an operation of a prediction system of FIG. 1.

FIG. 6 is a flowchart illustrating an example of an operation of a prediction system of FIG. 1.

FIG. 7 is a diagram for describing data used in a prediction system of FIG. 1.

FIG. 8 is a block diagram illustrating an example of an ensemble prediction model of FIG. 2.

FIG. 9 is a diagram for describing an operation of a long/short-term time-series generation unit of FIG. 8.

FIG. 10 is a block diagram illustrating an example of a trend extraction unit of FIG. 8.

FIG. 11 is a diagram for describing an operation of a trend extraction unit of FIG. 8.

FIG. 12 is a graph for describing a trend extraction unit.

FIG. 13 is a block diagram illustrating an example of a goodness-of-fit evaluation unit of FIG. 8.

FIG. 14 is a diagram illustrating an operation of an error learning unit of FIG. 8.

FIG. 15 is a block diagram illustrating an example of a prediction system of FIG. 1.

### DETAILED DESCRIPTION

Below, embodiments of the present disclosure will be described in detail and clearly to such an extent that one skilled in the art easily carries out the present disclosure.

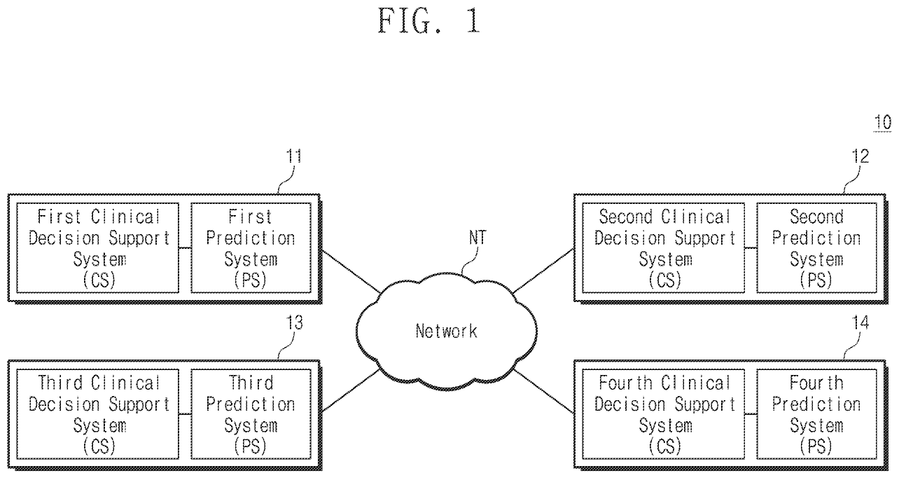

FIG. 1 is a block diagram illustrating a health state prediction system according to an embodiment of the present disclosure. Referring to FIG. 1, a health state prediction system 10 may include first to fourth medical support systems 11 to 14, and a network NT. Each of the first to fourth medical support systems 11 to 14 may include a clinical decision support system CS and a prediction system PS.

For example, the first to fourth medical support systems 11 to 14 may be respectively provided in different medical institutions or public institutions. Each of different medical institutions or public institutions may predict a health state of a future time point of the user by individually training a prediction model and applying medical data of the user to the prediction model built by the learning.

For example, the first medical support system 11 may include a first prediction system PS and a first clinical decision support system CS; the second medical support system 12 may include a second prediction system PS and a second clinical decision support system CS; the third medical support system 13 may include a third prediction system PS and a third clinical decision support system CS; the fourth medical support system 14 may include a fourth prediction system PS and a fourth clinical decision support system CS.

Each of the first to fourth medical support systems 11 to 14 may refer to a system for supporting a doctor's treatment in a medical institution. Each of the first to fourth prediction systems PS may be provided with medical data (e.g., health data or health time-series data) from the corresponding clinical decision support system CS. For example, the first prediction system PS may receive medical data including a health history of a patient from the first clinical decision support system CS.

In an embodiment, each of the first to fourth prediction systems PS may predict future health information based on the received medical data and may provide the future health information to the corresponding clinical decision support system CS. For example, the first prediction system PS may predict a patient's health state at a future time point by using the medical data. The first prediction system PS may send the predicted future health information or future health state to the first clinical decision support system CS.

In an embodiment, the first to fourth prediction systems PS may interwork with each other over the network NT. For example, the first prediction system PS may communicate with the second to fourth prediction systems PS over the network NT. That is, the first prediction system PS may exchange data with the second to fourth prediction systems PS over the network NT.

For example, the first medical support system 11 may send a prediction result request to the remaining medical support systems 12 to 14. The first medical support system 11 may receive a plurality of external prediction results from the remaining medical support systems 12 to 14. Because the plurality of external prediction results are obtained based on different prediction models, the plurality of external prediction results may have different values. The reason is that the first to fourth medical support systems 11 to 14 train and build respective prediction models based on different time-series medical data, that is, different training data. Due to sensitive characteristics of medical data such as an ethical issue, a legal issue, and a personal privacy issue, it is difficult to share data for each medical institution, and it is difficult to make big data. Accordingly, in building individual prediction models, the first to fourth medical support systems 11 to 14 may ensemble prediction results, and thus, it may be possible to predict a future health in consideration of various data learning.

The first to fourth medical support systems 11 to 14 may analyze time-series data based on different prediction models. In an environment where data share and exchange is difficult due to the sensitivity of medical data, each medical institution or hospital may train a prediction model from a database built therein. Time-series medical data may be concentrated in a specific medical institution in terms of a characteristic of a medical environment. A hospital specializing in a specific disease may collect medical data concentrated in the specific disease. The range of time-series medical data may be concentrated in a specific medical institution due to a health state difference of a visiting patient group. Under the above situation, the health state prediction system 10 of the present disclosure may obtain and ensemble results from prediction models built in different manners and thus may provide the user with improved information for supporting the clinical decision.

The first prediction system PS according to an embodiment of the present disclosure may generate partial time-series data based on health time-series data of a patient. The first prediction system PS may send the partial time-series data and the prediction result request to the second to fourth medical support systems 12 to 14. The first prediction system PS may receive a plurality of external prediction results associated with the partial time-series data thus sent. The first prediction system PS may analyze a trend between the health time-series data and the plurality of external prediction results and may calculate an external prediction goodness-of-fit. The first prediction system PS may calculate an ensemble prediction result based on the external prediction goodness-of-fit.

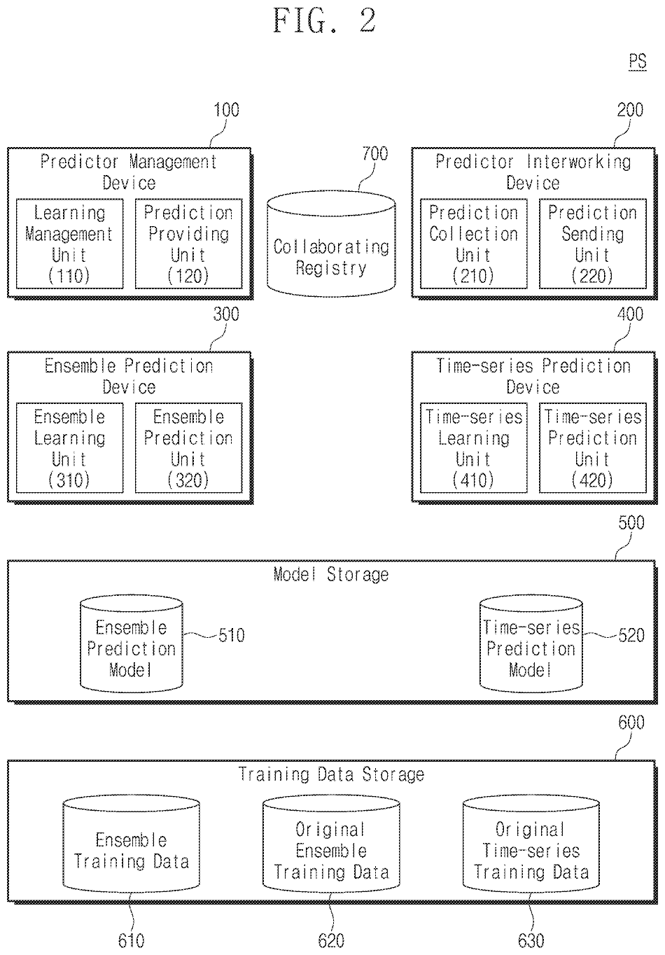

FIG. 2 is a block diagram illustrating an example of a prediction system of FIG. 1. Referring to FIGS. 1 and 2, the prediction system PS may include a predictor management device 100, a predictor interworking device 200, an ensemble prediction device 300, a time-series prediction device 400, model storage 500, training data storage 600, and a collaborating registry 700.

The predictor management device 100 may receive a prediction request and health data (or health time-series data) from the clinical decision support system CS. The predictor management device 100 may provide a prediction result to the clinical decision support system CS in response to the prediction request. For example, the prediction request may indicate a prediction result request for a health state of a future time point based on the provided health data.

In detail, the prediction request may include a time-series prediction request and an ensemble prediction request. The time-series prediction request may be used to request a result of predicting a health state of a future time point based on a time-series prediction model. The ensemble prediction request may be used to request a result of predicting a health state of a future time point based on an ensemble prediction model.

The predictor management device 100 may include a learning management unit 110 and a prediction providing unit 120.

The learning management unit 110 may receive a request for learning (i.e., a learning request) from a user interface (not illustrated) or a terminal (not illustrated). For example, the user interface may be configured to perform instruction, request, or data communication between the user and the learning management unit 110. That is, the user interface may provide the learning request to the learning management unit 110.

In an embodiment, the user interface may include a virtual device such as a command line interface (CLI), a graphic user interface (GUI), or a web user interface (WUI). The terminal may refer to an electronic device, which is capable of providing the learning request, such as a smartphone, a desktop, a laptop, or a wearable device. The terminal may provide the learning request to the learning management unit 110 over the network NT.

In an embodiment, the learning request may include a time-series learning request and an ensemble learning request. The time-series learning request may be used to request the learning of the time-series prediction model, and the ensemble learning request may be used to request the learning of the ensemble prediction model.

The learning management unit 110 may send a learning execution request to a prediction device in response to the learning request. The learning execution request may direct a learning execution start of a prediction model. For example, the learning management unit 110 may send the learning execution request to the time-series prediction device 400 in response to the time-series learning request. The learning management unit 110 may send the learning execution request to the ensemble prediction device 300 in response to the ensemble learning request.

The prediction providing unit 120 may send a prediction execution request to the prediction device in response to the prediction request. The prediction providing unit 120 may send a prediction result received from the prediction device to the clinical decision support system CS.

For example, the prediction providing unit 120 may send the prediction execution request to the time-series prediction device 400 in response to the time-series prediction request. The prediction providing unit 120 may send the time-series prediction result provided from the time-series prediction device 400 to the clinical decision support system CS.

The prediction providing unit 120 may send the prediction execution request to the ensemble prediction device 300 in response to the ensemble prediction request. The prediction providing unit 120 may provide the ensemble prediction result provided from the ensemble prediction device 300 to the clinical decision support system CS.

The predictor interworking device 200 may be configured to communicate with an external medical support system. For example, the predictor interworking device 200 of the first medical support system 11 may communicate with the second to fourth medical support systems 12 to 14. The predictor interworking device 200 of the first medical support system 11 may send the prediction result request to the second to fourth medical support systems 12 to 14 and may receive external prediction results from the second to fourth medical support systems 12 to 14. The prediction result request may be used to request a prediction result obtained by using a prediction model of each of external medical support systems.

The predictor interworking device 200 of the first medical support system 11 may receive the prediction result requests from the second to fourth medical support systems 12 to 14. The predictor interworking device 200 of the first medical support system 11 may send a generated prediction result to the second to fourth medical support systems 12 to 14 in response to the prediction result request.

The predictor interworking device 200 may include a prediction collection unit 210 and a prediction sending unit 220. The prediction collection unit 210 may send the prediction result request to external medical support systems and may receive external prediction results from the external medical support systems. That is, the prediction collection unit 210 may collect the external prediction results from the external medical support systems.

In an embodiment, the prediction collection unit 210 may receive an external prediction result request for original ensemble training data from an ensemble learning unit 310. The prediction collection unit 210 may load original ensemble training data 620 from the training data storage 600 in response to the external prediction result request. That is, the prediction collection unit 210 may send an original ensemble training data request to the training data storage 600 and may receive the original ensemble training data from the training data storage 600.

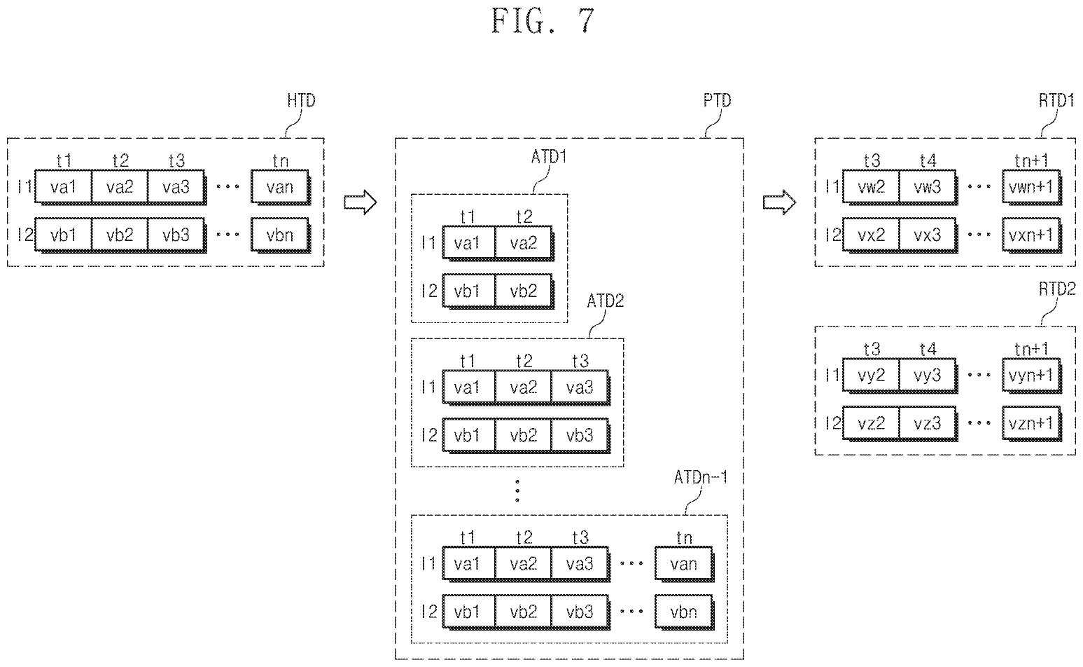

The prediction collection unit 210 may perform a partial time-series conversion operation based on the original ensemble training data and may generate partial time-series data. The partial time-series conversion operation will be described with to FIG. 7. The prediction collection unit 210 may send the prediction result request and the partial time-series data to external medical support systems registered at the collaborating registry 700. The prediction collection unit 210 may receive a prediction result (or a plurality of external prediction results) from the registered external medical support systems. The plurality of external prediction results may be understood as a result of predicting a health state of a predicted time based on the partial time-series data.

The prediction collection unit 210 may generate ensemble training data based on the received external prediction results and an original time-series health record (or original ensemble training data). That is, the prediction collection unit 210 may generate the ensemble training data based on the plurality of external prediction results and the original ensemble training data. The prediction collection unit 210 may store the generated ensemble training data in the training data storage 600.

In an embodiment, the prediction collection unit 210 may receive the external prediction result request for health time-series data from an ensemble prediction unit 320. The prediction collection unit 210 may send the prediction result request and the health time-series data (or partial time-series data of the health time-series data) to the registered external medical support systems in response to the external prediction result request. The prediction collection unit 210 may receive a plurality of external prediction results from the registered external medical support systems. The prediction collection unit 210 may merge the plurality of external prediction results and the health time-series data thus received. The prediction collection unit 210 may send the merged data to the ensemble prediction unit 320.

The ensemble prediction device 300 may predict a health state of a future time point based on the health time-series data, by using the ensemble prediction model. For example, the ensemble prediction device 300 may input the health time-series data to the ensemble prediction model. The ensemble prediction device 300 may generate and provide a prediction result associated with a health state of a future time point.

The ensemble prediction device 300 may include the ensemble learning unit 310 and the ensemble prediction unit 320. The ensemble learning unit 310 may train an ensemble prediction model 510 based on ensemble training data 610. The ensemble prediction model 510 may be built through an artificial neural network, deep learning, or machine learning.

In an embodiment, the ensemble learning unit 310 may receive the learning execution request from the learning management unit 110. The ensemble learning unit 310 may send the external prediction result request for original ensemble training data to the predictor interworking device 200 in response to the learning execution request. The ensemble learning unit 310 may train the ensemble prediction model based on the ensemble training data 610 thus generated and may store the ensemble prediction model in the model storage 500.

The ensemble prediction unit 320 may analyze a plurality of external prediction results corresponding to a specific user (e.g., a patient) based on the ensemble prediction model 510 trained by the ensemble learning unit 310 and may generate an ensemble prediction result. In an embodiment, the ensemble prediction unit 320 may receive the prediction execution request and the health time-series data from the prediction providing unit 120.

In an embodiment, the ensemble prediction unit 320 may send the external prediction execution request for the health time-series data to the predictor interworking device 200 in response to the prediction execution request. The ensemble prediction unit 320 may receive the merged data from the predictor interworking device 200. The merged data may be generated based on a plurality of external prediction results associated with the health time-series data and the health time-series data. The ensemble prediction unit 320 may input the merged data to the ensemble prediction model 510 to calculate an ensemble prediction result. The ensemble prediction unit 320 may send the calculated ensemble prediction result to the predictor management device 100.

The time-series prediction device 400 may predict a health state of a future time point based on the health time-series data, by using the time-series prediction model. For example, the time-series prediction device 400 may input the health time-series data to the time-series prediction model. The time-series prediction device 400 may generate and provide a prediction result associated with a health state of a future time point.

The time-series prediction device 400 may include a time-series learning unit 410 and a time-series prediction unit 420. The time-series learning unit 410 may receive the learning execution request from the learning management unit 110. The time-series learning unit 410 may create a time-series prediction model based on original time-series training data in response to the learning execution request and may store the time-series prediction model in the model storage 500.

The time-series prediction unit 420 may receive the prediction execution request from the prediction providing unit 120. The time-series prediction unit 420 may calculate a time-series prediction result by inputting health time-series data to the time-series prediction model in response to the prediction execution request. The time-series prediction unit 420 may send the calculated time-series prediction result to the predictor management device 100.

The model storage 500 may store the ensemble prediction model 510 and a time-series prediction model 520. The training data storage 600 may store the ensemble training data 610, the original ensemble training data 620, and original time-series training data 630. The original ensemble training data 620 may include time-series medical data for training the ensemble prediction model 510. The original time-series training data 630 may include time-series medical data for training the time-series prediction model 520.

The original ensemble training data 620 or the original time-series training data 630 may include time-series medical data indicating a user health state obtained based on diagnosis, treatment, examination, or medication prescription. The time-series data may include features respectively corresponding to a plurality of times. For example, the time-series medical data may be EMR (Electronic Medical Record) data or PHR (Personal Health Record) data.

The ensemble training data 610 may include data that are generated by merging the original ensemble training data 620 and external prediction results provided from external medical support systems. The prediction results provided from the external medical support systems may indicate prediction results associated with original ensemble training data or partial time-series data of the original ensemble training data. The ensemble training data 610, the original ensemble training data 620, and the original time-series training data 630 may be organized in a server or a storage medium.

The collaborating registry 700 may store information about external medical support systems for ensemble prediction. Each component of the prediction system PS may be implemented with hardware or may be implemented with firmware, software, or a combination thereof. For example, the software (or firmware) may be loaded onto a memory (not illustrated) included in the prediction system PS and may be executed by a processor (not illustrated). Each component of the prediction system PS may be implemented with a dedicated logic circuit such as a field programmable gate array (FPGA) or an application specific integrated circuit (ASIC).

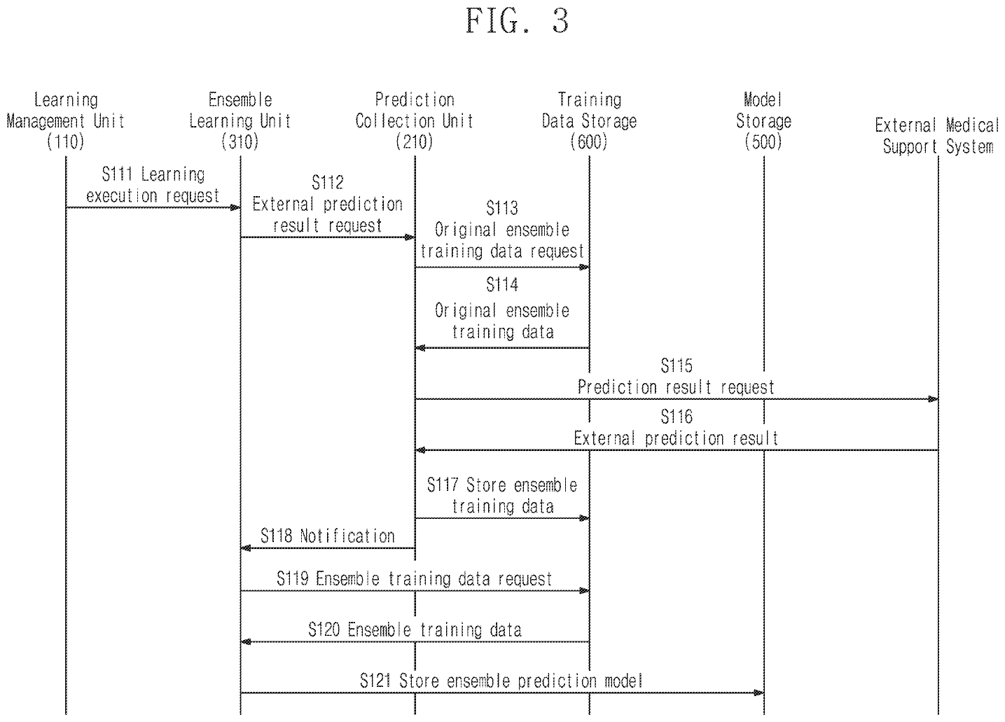

FIG. 3 is a flowchart illustrating an example of an operation of a prediction system of FIG. 1. An ensemble learning operation will be described with reference to FIGS. 1, 2, and 3. The prediction system PS may receive the ensemble learning request from a user interface or a terminal. The prediction system PS may create the ensemble prediction model in response to the received ensemble learning request and may store the ensemble prediction model in the model storage 500.

In operation S111, the learning management unit 110 may send the learning execution request to the ensemble learning unit 310. For example, the learning management unit 110 may send the learning execution request to the ensemble learning unit 310 in response to the ensemble learning request provided from the user interface or the terminal.

In operation S112, the ensemble learning unit 310 may send the external prediction result request to the prediction collection unit 210. For example, the ensemble learning unit 310 may receive the learning execution request from the learning management unit 110. The ensemble learning unit 310 may send the external prediction result request for original ensemble training data to the prediction collection unit 210 in response to the learning execution request.

In operation S113, the prediction collection unit 210 may send the original ensemble training data request to the training data storage 600. For example, the prediction collection unit 210 may receive the external prediction result request for the original ensemble training data from the ensemble learning unit 310. The prediction collection unit 210 may send the original ensemble training data request for loading the original ensemble training data to the training data storage 600 in response to the external prediction result request.

In operation S114, the training data storage 600 may send the original ensemble training data to the prediction collection unit 210. The training data storage 600 may receive the original ensemble training data request from the prediction collection unit 210. The training data storage 600 may send the original ensemble training data to the prediction collection unit 210 in response to the original ensemble training data request.

In operation S115, the prediction collection unit 210 may send the prediction result request to an external medical support system. The prediction collection unit 210 may perform the partial time-series data conversion operation on the received original ensemble training data. The prediction collection unit 210 may generate the partial time-series data through the partial time-series data conversion operation. The prediction collection unit 210 may send the partial time-series data and the prediction result request to the external medical support system over the network NT.

For brevity of drawing, an example in which the prediction collection unit 210 sends the prediction result request to one external medical support system is illustrated in FIG. 3, but the present disclosure is not limited thereto. For example, the prediction collection unit 210 may send the prediction result request to a plurality of external medical support systems. For example, the prediction collection unit 210 of the first medical support system 11 may send the prediction result request to the second to fourth medical support systems 12 to 14.

In operation S116 is, the prediction collection unit 210 may receive an external prediction result from the external medical support system. For example, the prediction collection unit 210 may receive the external prediction result for the original ensemble training data from the external medical support system over the network NT.

In an embodiment, the prediction collection unit 210 may receive a plurality of external prediction results from the plurality of external medical support systems. For example, the prediction collection unit 210 of the first medical support system 11 may receive a plurality of external prediction results from the second to fourth medical support systems 12 to 14.

In operation S117, the prediction collection unit 210 may store the ensemble training data in the training data storage 600. For example, the prediction collection unit 210 may merge the received external prediction result and the original ensemble training data to generate the ensemble training data. The prediction collection unit 210 may send the ensemble training data to the training data storage 600.

In operation S118, the prediction collection unit 210 may send a notification to the ensemble learning unit 310. For example, the prediction collection unit 210 may receive the prediction result from the external medical support system and may send, to the ensemble learning unit 310, the notification indicating that the ensemble training data are stored in the training data storage 600.

In operation S119, the ensemble learning unit 310 may send the ensemble training data request to the training data storage 600. For example, to load the ensemble training data, the ensemble learning unit 310 may send the ensemble training data request to the training data storage 600.

In operation S120, the training data storage 600 may send the ensemble training data to the ensemble learning unit 310. For example, the training data storage 600 may receive the ensemble training data request from the ensemble learning unit 310. The training data storage 600 may send the stored ensemble training data to the ensemble learning unit 310 in response the ensemble training data request.

In operation S121, the ensemble learning unit 310 may store the ensemble prediction model in the model storage 500. For example, the ensemble learning unit 310 may create or train the ensemble prediction model based on the ensemble training data. The ensemble learning unit 310 may send the created ensemble prediction model to the model storage 500.

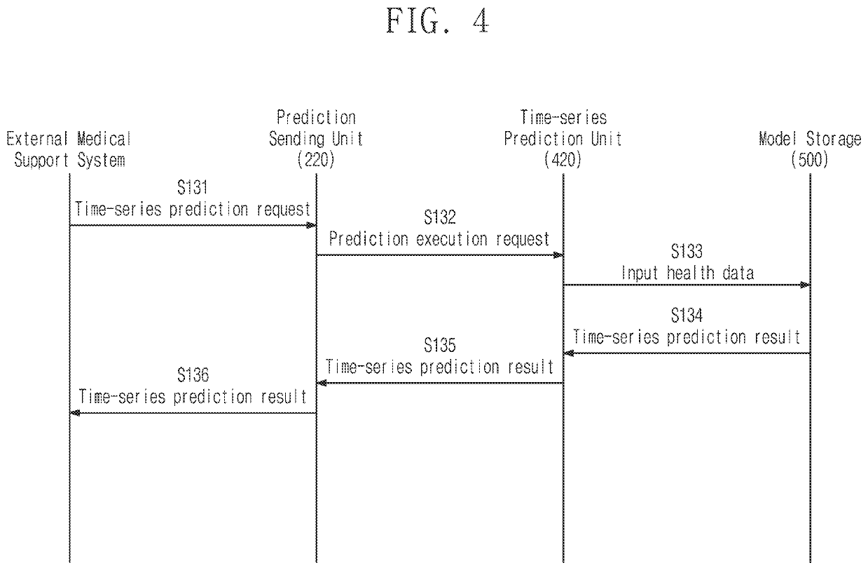

FIG. 4 is a flowchart illustrating an example of an operation of a prediction system of FIG. 1. A time-series prediction operation will be described with reference to FIGS. 1, 2, and 4. In operation S131, the prediction sending unit 220 may receive the time-series prediction request from an external medical support system. For example, the prediction sending unit 220 may receive the time-series prediction request and health data (or partial time-series data) from the external medical support system over the network NT.

In operation S132, the prediction sending unit 220 may send the prediction execution request to the time-series prediction unit 420. For example, in response to the time-series prediction request, the prediction sending unit 220 may send the prediction execution request and the health data received from the external medical support system to the time-series prediction unit 420.

In operation S133, the time-series prediction unit 420 may input the health data to the time-series prediction model 520. For example, in response to the received prediction execution request, the time-series prediction unit 420 may send the health data to the model storage 500 such that the health data are input to the time-series prediction model 520.

In operation S134, the model storage 500 may send a time-series prediction result to the time-series prediction unit 420. For example, the time-series prediction model 520 of the model storage 500 may calculate the time-series prediction result based on the received health data. The time-series prediction model 520 may send the time-series prediction result to the time-series prediction unit 420.

In operation S135, the time-series prediction unit 420 may send the time-series prediction result to the prediction sending unit 220. For example, the time-series prediction unit 420 may send the time-series prediction result provided from the time-series prediction model 520 to the prediction sending unit 220.

In operation S136, the prediction sending unit 220 may send the time-series prediction result to the external medical support system. For example, the prediction sending unit 220 may send the time-series prediction result to the external medical support system, which sends the time-series prediction request, over the network NT.

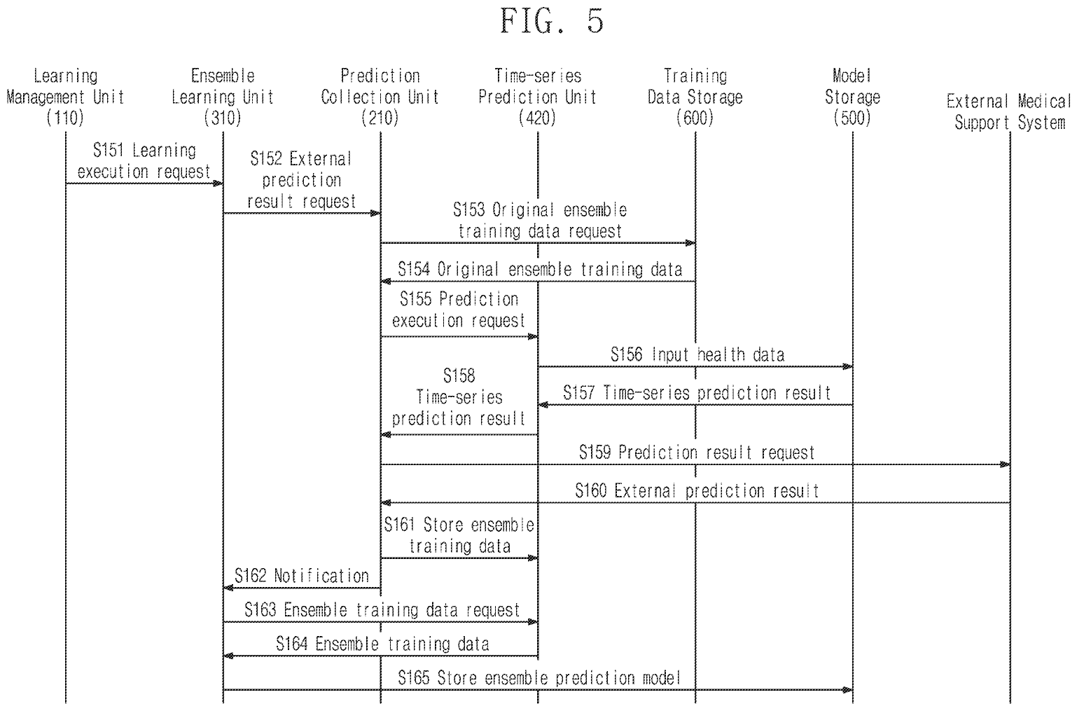

FIG. 5 is a flowchart illustrating an example of an operation of a prediction system of FIG. 1. An ensemble learning process using the time-series prediction result will be described with reference to FIGS. 1, 2, and 5. The prediction system PS may receive the ensemble learning request from a user interface or a terminal. The prediction system PS may create the ensemble prediction model in response to the received ensemble learning request and may store the ensemble prediction model in the model storage 500.

In operation S151, the learning management unit 110 may send the learning execution request to the ensemble learning unit 310. For example, the learning management unit 110 may send the learning execution request to the ensemble learning unit 310 in response to the ensemble learning request provided from the user interface or the terminal. Compared to the learning execution request of FIG. 3, the learning execution request of FIG. 5 may refer to an ensemble learning execution request using a time-series prediction result.

In operation S152, the ensemble learning unit 310 may send the external prediction result request to the prediction collection unit 210. For example, the ensemble learning unit 310 may receive the learning execution request from the learning management unit 110. The ensemble learning unit 310 may send the external prediction result request for original ensemble training data to the prediction collection unit 210 in response to the learning execution request. Compared to the external prediction result request of FIG. 3, the external prediction result request of FIG. 5 may include a prediction result request for the external medical support system and a prediction result request for the time-series prediction unit 420.

In operation S153, the prediction collection unit 210 may send the original ensemble training data request to the training data storage 600. For example, the prediction collection unit 210 may receive the external prediction result request for the original ensemble training data from the ensemble learning unit 310. The prediction collection unit 210 may send the original ensemble training data request for loading the original ensemble training data to the training data storage 600 in response to the external prediction result request.

In operation S154, the training data storage 600 may send the original ensemble training data to the prediction collection unit 210. The training data storage 600 may receive the original ensemble training data request from the prediction collection unit 210. The training data storage 600 may send the original ensemble training data to the prediction collection unit 210 in response to the original ensemble training data request.

In operation S155, the prediction collection unit 210 may send the prediction execution request to the time-series prediction unit 420. For example, the prediction collection unit 210 may send the prediction execution request and health data (or partial time-series data) to the time-series prediction unit 420. The prediction collection unit 210 may perform the partial time-series data conversion operation on the received original ensemble training data. The prediction collection unit 210 may generate the partial time-series data through the partial time-series data conversion operation.

In operation S156, the time-series prediction unit 420 may input the health data to the time-series prediction model 520. For example, in response to the received prediction execution request, the time-series prediction unit 420 may send the health data to the model storage 500 such that the health data are input to the time-series prediction model 520.

In operation S157, the model storage 500 may send a time-series prediction result to the time-series prediction unit 420. For example, the time-series prediction model 520 of the model storage 500 may calculate the time-series prediction result based on the received health data. The time-series prediction model 520 may send the time-series prediction result to the time-series prediction unit 420.

In operation S158, the time-series prediction unit 420 may send the time-series prediction result to the prediction collection unit 210. For example, the time-series prediction unit 420 may send the time-series prediction result provided from the time-series prediction model 520 to the prediction collection unit 210.

In operation S159, the prediction collection unit 210 may send the prediction result request to an external medical support system. The prediction collection unit 210 may perform the partial time-series data conversion operation on the received original ensemble training data. The prediction collection unit 210 may generate the partial time-series data through the partial time-series data conversion operation. The prediction collection unit 210 may send the partial time-series data and the prediction result request to the external medical support system over the network NT.

For brevity of drawing, an example in which the prediction collection unit 210 sends the prediction result request to one external medical support system is illustrated in FIG. 5, but the present disclosure is not limited thereto. For example, the prediction collection unit 210 may send the prediction result request to a plurality of external medical support systems.

In operation S160 is, the prediction collection unit 210 may receive an external prediction result from the external medical support system. For example, the prediction collection unit 210 may receive the prediction result for the original ensemble training data from the external medical support system over the network NT. In an embodiment, the prediction collection unit 210 may receive a plurality of external prediction results from the plurality of external medical support systems.

An example in which operation S159 and operation S160 are performed after operation S155 to operation S158 is illustrated in FIG. 5, but the present disclosure is not limited thereto. For example, operation S159 and operation S160 may be performed before operation S155 to operation S158 or at the same time with operation S155 to operation S158.

In operation S161, the prediction collection unit 210 may store the ensemble training data in the training data storage 600. For example, the prediction collection unit 210 may merge the received prediction result and the original ensemble training data to generate the ensemble training data. The received prediction result may include the external prediction result received from the external medical support system and the time-series prediction result received from the time-series prediction unit 420. The prediction collection unit 210 may send the ensemble training data to the training data storage 600.

In operation S162, the prediction collection unit 210 may send a notification to the ensemble learning unit 310. For example, the prediction collection unit 210 may receive the prediction result (e.g., the external prediction result and the time-series prediction result) from the external medical support system and the time-series prediction unit 420 and may send, to the ensemble learning unit 310, the notification indicating that the ensemble training data are stored in the training data storage 600.

In operation S163, the ensemble learning unit 310 may send the ensemble training data request to the training data storage 600. For example, to load the ensemble training data, the ensemble learning unit 310 may send the ensemble training data request to the training data storage 600.

In operation S164, the training data storage 600 may send the ensemble training data to the ensemble learning unit 310. For example, the training data storage 600 may receive the ensemble training data request from the ensemble learning unit 310. The training data storage 600 may send the stored ensemble training data to the ensemble learning unit 310 in response the ensemble training data request.

In operation S165, the ensemble learning unit 310 may store the ensemble prediction model in the model storage 500. For example, the ensemble learning unit 310 may train the ensemble prediction model based on the ensemble training data. The ensemble learning unit 310 may send the ensemble prediction model to the model storage 500.

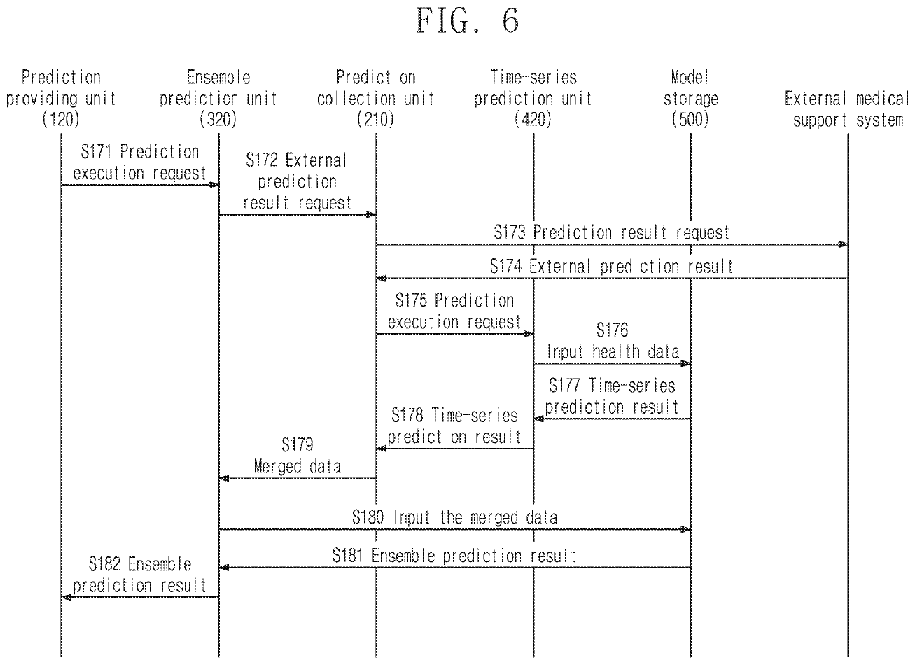

FIG. 6 is a flowchart illustrating an example of an operation of a prediction system of FIG. 1. An ensemble prediction operation using a time-series prediction result will be described with reference to FIGS. 1, 2, and 6. The prediction system PS may receive the ensemble prediction request and health data from the clinical decision support system CS. The ensemble prediction request may refer to an ensemble prediction request using an external prediction result and a time-series prediction result.

In response to the received ensemble learning request, the prediction system PS may receive the external prediction result associated with the health data and may calculate the time-series prediction result by using the time-series prediction model. The prediction system PS may calculate the ensemble prediction result by inputting the health data, the external prediction result, and the time-series prediction result to the ensemble prediction model.

In operation S171, the prediction providing unit 120 may send the prediction execution request to the ensemble prediction unit 320. For example, the prediction providing unit 120 may send the prediction execution request and the received health data to the ensemble prediction unit 320 in response to the ensemble prediction request provided from the clinical decision support system CS.

In operation S172, the ensemble prediction unit 320 may send the external prediction result request to the prediction collection unit 210. For example, the ensemble prediction unit 320 may receive the prediction execution request from the prediction providing unit 120. The ensemble prediction unit 320 may send the external prediction result request for health data and the health data to the prediction collection unit 210 in response to the prediction execution request.

In operation S173, the prediction collection unit 210 may send the prediction result request to an external medical support system. For example, the prediction collection unit 210 may perform the partial time-series data conversion operation on the received health data to generate partial time-series data. The prediction collection unit 210 may send the health data (or partial time-series data) and the prediction result request to the external medical support system over the network NT.

For brevity of drawing, an example in which the prediction collection unit 210 sends the prediction result request to one external medical support system is illustrated in FIG. 6, but the present disclosure is not limited thereto. For example, the prediction collection unit 210 may send the prediction result request to a plurality of external medical support systems.

In operation S174 is, the prediction collection unit 210 may receive an external prediction result from the external medical support system. For example, the prediction collection unit 210 may receive the prediction result associated with the health data from the external medical support system over the network NT. In an embodiment, the prediction collection unit 210 may receive a plurality of prediction results from the plurality of external medical support systems.

In operation S175, the prediction collection unit 210 may send the prediction execution request to the time-series prediction unit 420. For example, the prediction collection unit 210 may send the prediction execution request and health data (or partial time-series data) to the time-series prediction unit 420.

In operation S176, the time-series prediction unit 420 may input the health data to the time-series prediction model 520. For example, in response to the received prediction execution request, the time-series prediction unit 420 may send the health data to the model storage 500 such that the health data are input to the time-series prediction model 520.

In operation S177, the model storage 500 may send a time-series prediction result to the time-series prediction unit 420. For example, the time-series prediction model 520 of the model storage 500 may calculate the time-series prediction result based on the received health data. The time-series prediction model 520 may send the time-series prediction result to the time-series prediction unit 420.

In operation S178, the time-series prediction unit 420 may send the time-series prediction result to the prediction collection unit 210. For example, the time-series prediction unit 420 may send the time-series prediction result provided from the time-series prediction model 520 to the prediction collection unit 210.

An example in which operation S175 to operation S178 are performed after operation S173 and operation S174 is illustrated in FIG. 6, but the present disclosure is not limited thereto. For example, operation S175 to operation S178 may be performed before operation S173 and operation S174 or at the same time with operation S173 and operation S174.

In operation S179, the prediction collection unit 210 may send the merged data to the ensemble prediction unit 320. For example, the prediction collection unit 210 may generate merged data based on the external prediction result, the time-series prediction result, and the health data. The prediction collection unit 210 may send the merged data to the ensemble prediction unit 320.

In operation S180, the ensemble prediction unit 320 may input the merged data to the ensemble prediction model 510 of the model storage 500. For example, the ensemble prediction unit 320 may receive the merged data from the prediction collection unit 210. For example, to calculate the ensemble prediction result, the ensemble prediction unit 320 may send the merged data to the model storage 500 such that the merged data are input to the ensemble prediction model 510.

In operation S181, the model storage 500 may send the ensemble prediction result to the ensemble prediction unit 320. For example, the ensemble prediction model 510 of the model storage 500 may calculate the ensemble prediction result based on the received merged data. The ensemble prediction model 510 may send the ensemble prediction result to the ensemble prediction unit 320.

In operation S182, the ensemble prediction unit 320 may send the ensemble prediction result to the prediction providing unit 120. For example, the ensemble prediction unit 320 may send the ensemble prediction result provided from the ensemble prediction model 510 to the prediction providing unit 120.

FIG. 7 is a diagram for describing data used in a prediction system of FIG. 1. Referring to FIGS. 1, 2, and 7, for brevity of drawing and convenience of description, it is assumed that the prediction system PS receives external prediction results RTD1 and RTD2 from two external medical support systems among a plurality of external medical support systems.

For example, it is assumed that the prediction system PS is a first prediction system of the first medical support system 11, the external medical support systems include the second medical support system 12 and the third medical support system 13, the first external prediction result RTD1 is sent from the second medical support system 12, and the second external prediction result RTD2 is sent from the third medical support system 13.

Health time-series data HTD, partial time-series data PTD, and the external prediction results RTD1 and RTD2 may have the format of time-series data TD. The time-series data TD may include features corresponding to a plurality of time points and a plurality of items. For example, the items may represent various health indicators such as a blood pressure, a blood sugar, a cholesterol level, and a weight. The features may represent values of respective items diagnosed, tested, or prescribed at a particular time.

It is assumed that the health time-series data HTD includes features va1 to van and vb1 to vbn corresponding to first to n-th time points t1 to tn and first and second items I1 and I2. The partial time-series data PTD associated with the health time-series data HTD may be generated based on the health time-series data HTD. The partial time-series data PTD may refer to a portion of the health time-series data HTD. The partial time-series data PTD may include features corresponding to arbitrary continuous time points among all the time points of the health time-series data HTD.

The partial time-series data PTD may include accumulation time-series data. The accumulation time-series data may be generated by accumulating features of previous time points of each of the plurality of time points t1 to tn with regard to the health time-series data HTD. A first accumulation time-series data ATD1 may be generated by accumulating features of time points before the third time point t3, a second accumulation time-series data ATD2 may be generated by accumulating features of time points before the fourth time point t4, and a (n−1)-th accumulation time-series data ATDn−1 may be generated by accumulating features of time points before a (n+1)-th time point tn+1. The partial time-series data PTD may include the first to (n−1)-th accumulation time-series data ATD1 to ATDn−1.

Each of the external medical support systems may analyze the first to (n−1)-th accumulation time-series data ATD1 to ATDn−1 −to generate prediction features corresponding to the third to (n+1−)-th time points t3 to tn+1. That is, the prediction system PS may generate various accumulation time-series data ATD1 to ATDn−1 by using the health time-series data HTD, which allows the external medical support systems to generate external prediction results at various time points.

The first external prediction result RTD1 may be generated from the second medical support system 12 based on the accumulation time-series data ATD1 to ATDn−1. The second external prediction result RTD2 may be generated from the third medical support system 13 based on the accumulation time-series data ATD1 to ATDn−1. Each of the external medical support systems 12 and 13 may analyze the first accumulation time-series data ATD1 to generate prediction features corresponding to the third time point t3, may analyze the second accumulation time-series data ATD2 to generate prediction features corresponding to the fourth time point t4, and may analyze the (n−1)-th accumulation time-series data ATDn−11 to generate prediction features corresponding to the (n+1)-th time point tn+1.

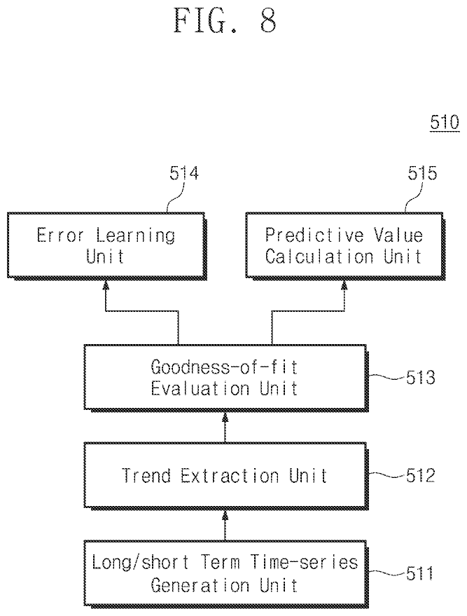

FIG. 8 is a block diagram illustrating an example of an ensemble prediction model of FIG. 2. Referring to FIGS. 2 and 8, the ensemble prediction model 510 may include a long/short-term time-series generation unit 511, a trend extraction unit 512, a goodness-of-fit evaluation unit 513, an error learning unit 514, and a predictive value calculation unit 515. Each component included in the ensemble prediction model 510 may be implemented with hardware or may be implemented with firmware, software, or a combination thereof.

The long/short-term time-series generation unit 511 may generate the partial time-series data PTD based on the time-series data TD. For example, the long/short-term time-series generation unit 511 may generate partial time-series data of an analysis length target with regard to the health time-series data HTD and the external prediction results RTD1 and RTD2. The partial time-series data PTD may include long-term time-series data LTD and short-term time-series data STD.

In an embodiment, the long/short-term time-series generation unit 511 may generate long-term time-series data LTD HTD for health time-series data and short-term time-series data STD_HTD for health time-series data. The long/short-term time-series generation unit 511 may generate long-term time-series data LTD_RTD1 for first external prediction result and short-term time-series data STD_RTD1 for first external prediction result. The long/short-term time-series generation unit 511 may generate long-term time-series data LTD_RTD2 for second external prediction result and short-term time-series data STD_RTD2 for second external prediction result.

The long-term time-series data LTD may include the long-term time-series data LTD_HTD for health time-series data, the long-term time-series data LTD_RTD1 for first external prediction result, and the long-term time-series data LTD_RTD2 for second external prediction result. The short-term time-series data STD may include the short-term time-series data STD_HTD for health time-series data, the short-term time-series data STD_RTD1 for first external prediction result, and the short-term time-series data STD_RTD2 for second external prediction result.

The trend extraction unit 512 may extract a plurality of trend features based on the long-term time-series data LTD and the short-term time-series data STD. The trend extraction unit 512 may generate feature windows by grouping each of the long-term time-series data LTD and the short-term time-series data STD at a window time interval. The trend extraction unit 512 may generate the feature windows by extracting prediction features, which belong to the window time interval from a target time, from each of the long-term time-series data LTD and the short-term time-series data STD.

For example, the target time may be one of the third to (n+1)-th time points t3 to tn+1 of FIG. 7. A feature window may include a plurality of window groups respectively corresponding to a plurality of target times. In an embodiment, when the window time interval is “3”, a window group whose target time is the fifth time point t5 may include prediction features corresponding to the third to fifth time points t3 to t5.

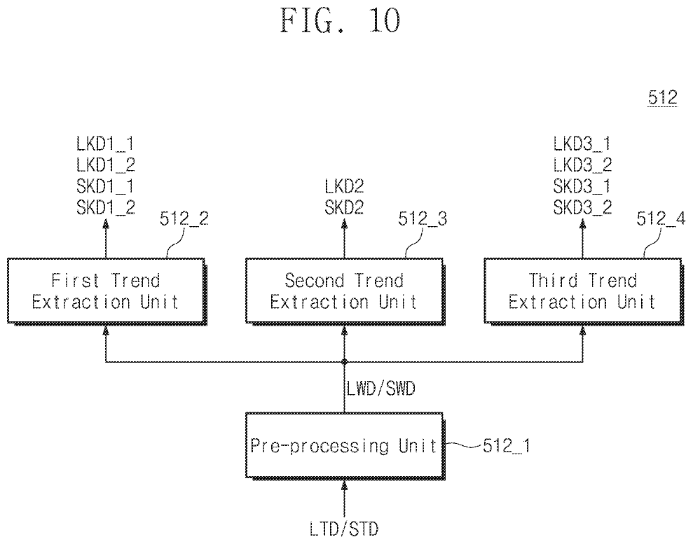

The trend extraction unit 512 may analyze a plurality of window groups of each of the long-term time-series data LTD and the short-term time-series data STD and may generate trends. The trend extraction unit 512 may extract a plurality of trends. A configuration and an operation method of the trend extraction unit 512 will be described in detail with reference to FIGS. 10 to 12.

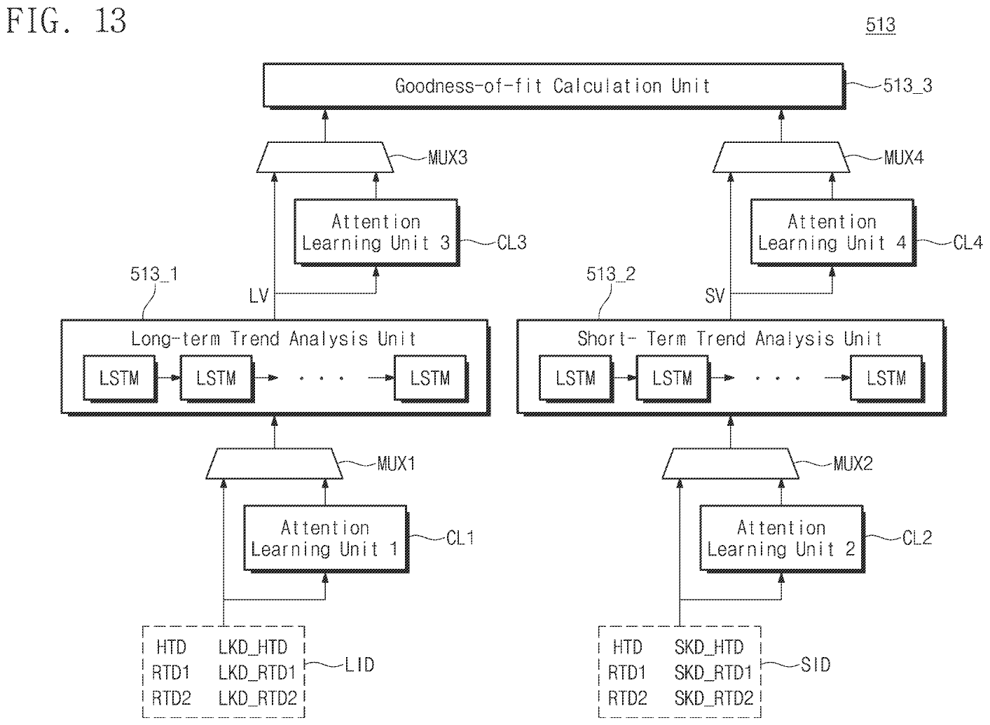

The goodness-of-fit evaluation unit 513 may evaluate and calculate time-series similarity and external prediction goodness-of-fit of the health time-series data HTD and the external prediction results RTD1 and RTD2, based on a plurality of trend features extracted from the trend extraction unit 512. A configuration and an operation method of the goodness-of-fit evaluation unit 513 will be described in detail with reference to FIG. 13.

The error learning unit 514 may receive the external prediction goodness-of-fit from the goodness-of-fit evaluation unit 513. The error learning unit 514 may calculate an error between the external prediction goodness-of-fit and real external goodness-of-fit calculated based on an experimental value of a prediction time. The error learning unit 514 may update an ensemble prediction model such that the error is minimized.

In an embodiment, when the trend extraction unit 512 and the goodness-of-fit evaluation unit 513 are implemented with an artificial neural network, the ensemble prediction model 510 may be trained in a back propagation manner. That is, the error learning unit 514 may adjust a parameter group for an operation of each component of the ensemble prediction model 510 in the back propagation manner.

The predictive value calculation unit 515 may receive the calculated external prediction goodness-of-fit from the goodness-of-fit evaluation unit 513. The predictive value calculation unit 515 may calculate an ensemble predictive value (or an ensemble prediction result) based on the external prediction goodness-of-fit. In an embodiment, the ensemble predictive value may be calculated based on an external prediction result, which has the greatest value of external prediction goodness-of-fit, from among a plurality of external prediction results. For example, the ensemble predictive value may be calculated by Equation 1 below.

Ensemble predictive value=p[argmax(pA, pN) [Equation 1]

In an embodiment, the ensemble predictive value may be calculated by adding results of multiplying respective predictive values pₖ of a plurality external prediction results and respective corresponding external prediction goodness-of-fit sₖ together. For example, the ensemble predictive value may be calculated by Equation 2 below.

$$Ensemble predictive value = {\sum }_{1}^{n} {p}_{k} \times {s}_{k} \qquad [ Equation 2 ]$$

In an embodiment, the predictive value calculation unit 515 may further include a linear regression layer. The linear regression layer may calculate the ensemble predictive value after learning the relationship between the ensemble predictive value and the external prediction goodness-of-fit, based on an external prediction result vector and an external prediction goodness-of-fit vector.

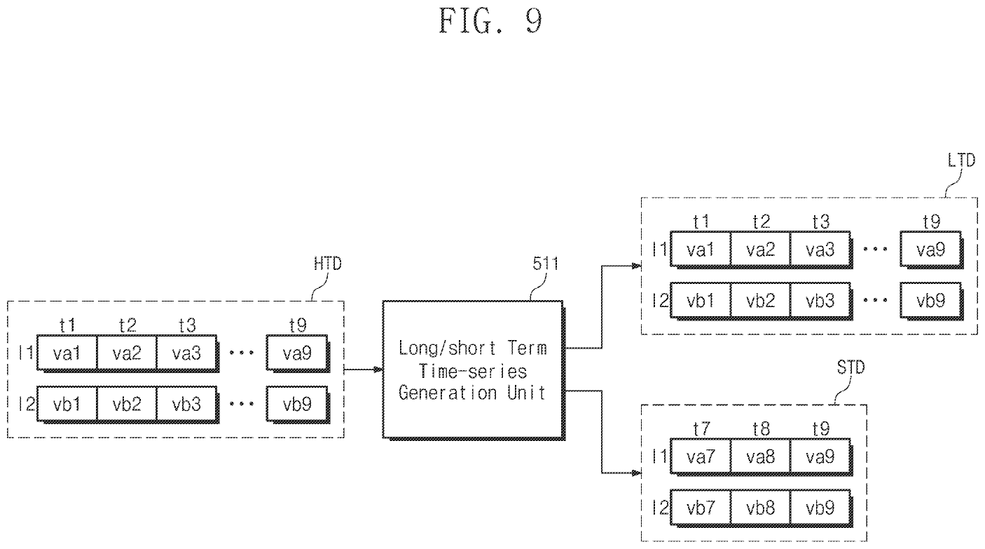

FIG. 9 is a diagram for describing an operation of a long/short-term time-series generation unit of FIG. 8. Referring to FIGS. 8 and 9, the long/short-term time-series generation unit 511 may generate the long-term time-series data LTD and the short-term time-series data STD based on the time-series data TD. For example, the long/short-term time-series generation unit 511 may receive the time-series data TD. As described above, the time-series data TD may include the health time-series data HTD, the first external prediction result RTD1, and the second external prediction result RTD2.

The long/short-term time-series generation unit 511 may generate the long-term time-series data LTD or the short-term time-series data STD based on the time-series data TD. The long-term time-series data LTD and the short-term time-series data STD may be the partial time-series data PTD of the time-series data TD. The long-term time-series data LTD and the short-term time-series data STD may include features of a plurality of time points continuous in the whole duration of the time-series data TD. The long/short-term time-series generation unit 511 may output the generated long-term time-series data LTD or the generated short-term time-series data STD to the trend extraction unit 512.

It is assumed that the time-series data TD include features va1 to va9 and vb1 to vb9 corresponding to a plurality of time points t1 to t9 and a plurality of items I1 and I2. It is assumed that the long-term time-series data LTD include features corresponding to the first to ninth time points t1 to t9. It is assumed that the short-term time-series data STD include features corresponding to the seventh to ninth time points t7 to t9.

The long-term time-series data LTD may refer to data for analyzing a long-term time-series feature. The long-term time-series data LTD may include features va1 to va9 and vb1 to vb9 of the first to ninth time points t1 to t9. For example, the long-term time-series data LTD may include features of the whole duration of the input time-series data TD. The long-term time-series data LTD may be the same as the input time-series data TD.

The short-term time-series data STD may refer to data for analyzing a short-term time-series feature. The short-term time-series data STD may include features va7 to va9 and vb7 to vb9 of the seventh to ninth time points t7 to t9. The short-term time-series data STD may include features of the input time-series data TD, which correspond to recent “b” time points (b being a natural number of 1 or more). For example, the short-term time-series data STD may include features, the number of which is less than the number of features of the input time-series data TD.

The long/short-term time-series generation unit 511 may adjust the number of features included in the short-term time-series data STD, the number of time points belonging to the whole duration of the short-term time-series data STD, or a size of the short-term time-series data STD. For example, the long/short-term time-series generation unit 511 may change a size of short-term time-series data depending on an analysis target duration. The long/short-term time-series generation unit 511 may generate the short-term time-series data STD including features of the eighth and ninth time points t8 and t9 belonging to a first analysis target duration and may generate the short-term time-series data STD including features of the sixth to ninth time points t6 to t9 belonging to a second analysis target duration,

In an embodiment, the long/short-term time-series generation unit 511 may generate one long-term time-series data LTD and a plurality of short-term time-series data STD. For example, the long/short-term time-series generation unit 511 may generate the long-term time-series data LTD including features of the first to ninths time points t1 to t9. The long/short-term time-series generation unit 511 may generate first short-term time-series data including features of the sixth to ninth time points t6 to t9, may generate second short-term time-series data including features of the seventh to ninth time points t7 to t9, and may generate third short-term time-series data including features of the eighth and ninth time points t8 and t9.

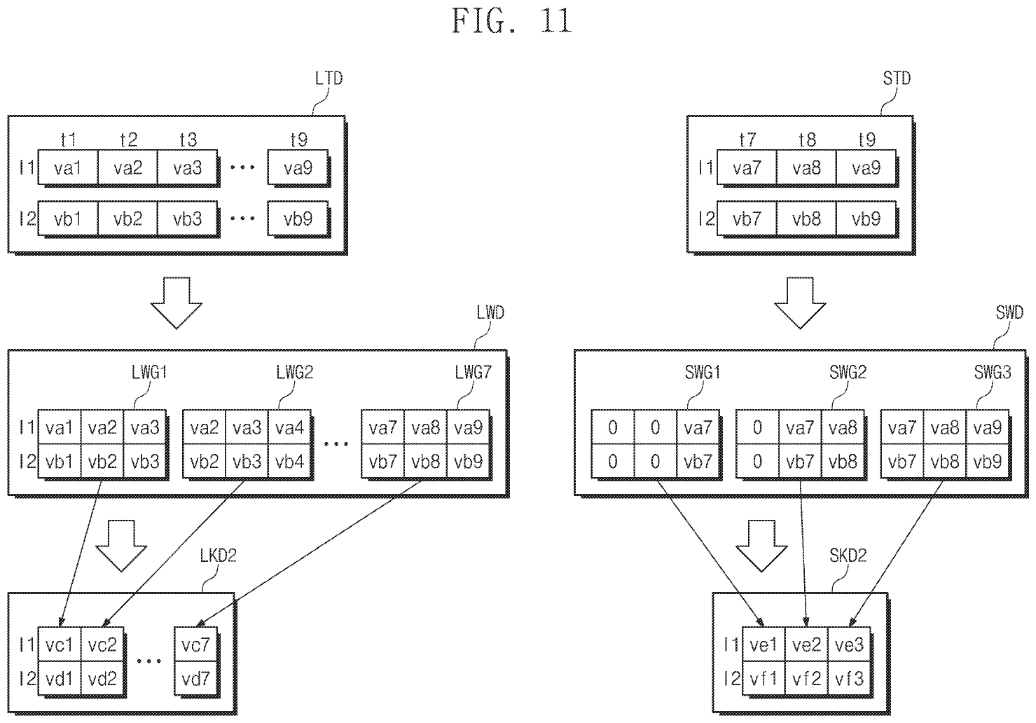

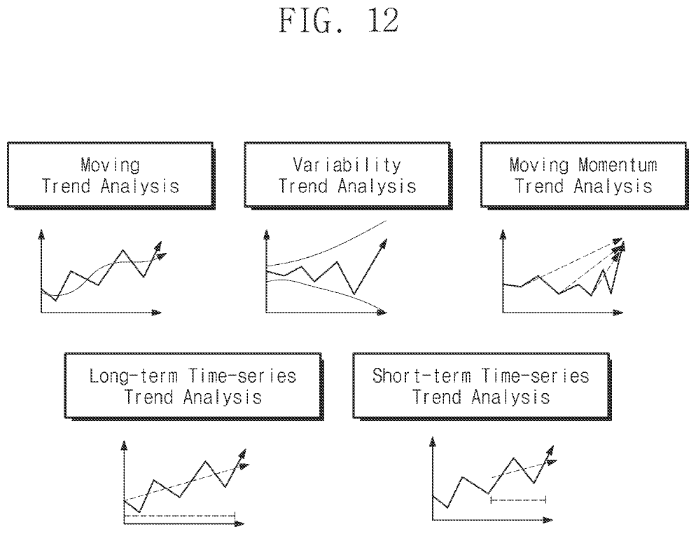

FIG. 10 is a block diagram illustrating an example of a trend extraction unit of FIG. 8. FIG. 11 is a diagram for describing an operation of a trend extraction unit of FIG. 8. FIG. 12 is a graph for describing a trend extraction unit. Referring to FIGS. 8, 10, and 11, the trend extraction unit 512 may include a pre-processing unit 512_1 and first to third trend extraction units 512_2 to 512_4. The trend extraction unit 512 may receive the long-term time-series data LTD and the short-term time-series data STD from the long/short-term time-series generation unit 511.

In detail, the trend extraction unit 512 may receive the long-term time-series data LTD_HTD for health time-series data, the short-term time-series data STD_HTD for health time-series data, the long-term time-series data LTD_RTD1 for first external prediction result, the short-term time-series data STD_RTD1 for the first external prediction result, the long-term time-series data LTD_RTD2 for second external prediction result, and the short-term time-series data STD_RTD2 for the second external prediction result.

The pre-processing unit 512_1 may generate a long-term feature window LWD by grouping the long-term time-series data LTD at a window time interval and may generate a short-term feature window SWD by grouping the short-term time-series data STD at a window time interval. The pre-processing unit 512_1 may generate the long-term feature window LWD by extracting features, which belong to a window time interval from a target time, from the long-term time-series data LTD and may generate the short-term feature window SWD by extracting features, which belong to a window time interval from a target time, from the short-term time-series data STD.

The long-term feature window LWD may include a plurality of long-term window groups corresponding to a plurality of target times, and the short-term feature window SWD may include a plurality of short-term window groups corresponding to a plurality of target times. For example, when a window time interval is “3” and a target time is a seventh time point t7, a window group may include features corresponding to the seventh to ninth time points t7 to t9.

In an embodiment, a long-term time-series trend may be used when the window time interval increases, and a short-term time-series trend may be used when the window time interval decreases. The window time interval may be adjusted depending on a purpose.

In an embodiment, the pre-processing unit 512_1 may generate a feature window vector to which a plurality of window time intervals are applied. That is, the pre-processing unit 512_1 may generate a long-term feature window vector and a short-term feature window vector.

For example, the pre-processing unit 512_1 may generate a first long-term feature window by grouping the long-term time-series data LTD at a first window time interval (e.g., 2) and may generate a second long-term feature window by grouping the long-term time-series data LTD at a second window time interval (e.g., 3). The pre-processing unit 512_1 may generate the long-term feature window vector including the first long-term feature window and the second long-term feature window. As in the above description, the pre-processing unit 512_1 may generate a short-term feature window vector.

As illustrated in FIG. 11, the pre-processing unit 512_1 may generate the long-term feature window LWD based on the long-term time-series data LTD and may generate the short-term feature window SWD based on the short-term time-series data STD. It is assumed that a window time interval is “3”. The pre-processing unit 512_1 may extract features corresponding to 3 time points and may generate feature windows.

For example, the pre-processing unit 512_1 may generate the long-term feature window LWD including first to seventh long-term window groups LWG1 to LWG7 based on the long-term time-series data LTD. The pre-processing unit 512_1 may generate the short-term feature window SWD including first to third short-term window groups SWG1 to SWG3 based on the short-term time-series data STD. Each of the window groups LWG1 to LWG7 and SWG1 to SWG3 may include features corresponding to three continuous time points. The window groups LWGI to LWG7 and SWG1 to SWG3 may be used to analyze a trend of features belonging to a window time interval.

In the case of the short-term time-series data STD, features corresponding to time points before the seventh time point t7 may not exist. In this case, values of empty time points (e.g., the fifth and sixth time points t5 and t6) of the first short-term window group SWG1 generated at the fifth time point t5 and a value of an empty time point (e.g., the sixth time point t6) of the second short-term window group SWG2 generated at the sixth time point t6 may be filled through the zero padding.

The first to third trend extraction units 512_2 to 512_4 may receive the long-term feature window LWD and the short-term feature window SWD provided from the pre-processing unit 512_1. The trend extraction unit 512 may extract a plurality of trends. For example, the first trend extraction unit 512_2 may extract a moving trend, the second trend extraction unit 512_3 may extract a variability trend, and the third trend extraction unit 512_4 may extract a moving momentum trend.

For example, the moving trend may be a vector expressing a gradual change trend of a value of time-series data. The variability trend may be a vector expressing a magnitude, a pattern, and a period of variability of a value in time-series data. The moving momentum trend may be a vector expressing a change direction including an increase and a decrease of time-series data, a strength for the change direction, and the like.

In an embodiment, the moving trend may include a moving feature and a trend transition feature. For example, the moving feature may use an indicator such as a moving average (MA) and a moving average convergence & divergence. For example, in Equation 3 below, aₖ represents a value of a coefficient corresponding to a k-th window group from among coefficients, xₖ represents the k-th window group. The moving average may be calculated by applying “1/k” to each of coefficients aₖ to aₖ like Equation 3 below.

MA=a₁xᵢ+a₂x₂, . . . +aₖxₖ [Equation 3]

In an embodiment, the trend transition feature may utilize a difference between window averages calculated while varying a window time interval “k”. When the condition that a first window time interval k1 is greater than a second window time interval k2, the case where a first trend extraction result extracted with the partial time series of the first window time interval k1 is smaller than a second trend extraction result extracted with the partial time series of the second window time interval k2 means the transition to an increasing trend, and the case where the first trend extraction result is greater than the second trend extraction result means the transition to a decreasing trend.

The variability trend may express a trend feature with the standard deviation and the variance for window groups. The moving momentum trend may include a slope feature and a variation feature. For example, the slope feature may be calculated by Equation 4 corresponding to a simple variation calculation equation. Alternatively, the slope feature may be calculated by Equation 5 corresponding to a slope equation of linear regression.

$$\frac{{y}_{k} - {y}_{1}}{{x}_{k -} {x}_{1}} \qquad [ Equation 4 ]$$

$$\frac{k ( \sum xy ) - ( \sum x ) ( \sum y )}{k ( \sum {x}^{2} ) - {( \sum x )}^{2}} \qquad [ Equation 5 ]$$

For example, the variation feature may be calculated by Equation 6 corresponding to a difference between two time points. The variation feature may be calculated by Equation 7 corresponding to a change ratio to a starting point.

$${x}_{k} - {x}_{1} \qquad [ Equation 6 ]$$

$$( \frac{{x}_{k}}{x 1} - 1 ) \cdot 100 \qquad [ Equation 7 ]$$

The first trend extraction unit 512_2 may generate a long-term moving feature trend LKD1_1 and a long-term trend transition feature trend LKD1_2 based on the long-term feature window LWD. The first trend extraction unit 512_2 may generate a short-term moving feature trend SKD1_1 and a short-term trend transition feature trend SKD1_2 based on the short-term feature window SWD.

The second trend extraction unit 512_3 may generate a long-term variability feature trend LKD2 based on the long-term feature window LWD. The second trend extraction unit 512_3 may generate a short-term variability feature trend SKD2 based on the short-term feature window SWD.

The third trend extraction unit 512_4 may generate a long-term slope feature trend LKD3_1 and a long-term variation feature trend LKD3_2 based on the long-term feature window LWD. The third trend extraction unit 512_4 may generate a short-term slope feature trend SKD3_1 and a short-term variation feature trend SKD3_2 based on the short-term feature window SWD.

For brevity of drawing and convenience of description, only one trend of a plurality of feature trends is illustrated in FIG. 11, and the remaining feature trends are omitted. That is, only the process in which the second trend extraction unit 512_3 generates the long-term variability feature trend LKD2 and the short-term variability feature trend SKD2 is illustrated.

In an embodiment, the second trend extraction unit 512_3 may analyze each of long-term window groups LWG1 to LWG7 included in the long-term feature window LWD and may generate a long-term variability feature trend LKD2. The long-term variability feature trend LKD2 may include trend features respectively corresponding to the long-term window groups LWG1 to LWG7.

For example, the second trend extraction unit 512_3 may analyze features va1 to va3 of a first item I1 in the first long-term window group LWG1 to generate a variability feature trend score vc1 and may analyze features vb1 to vb3 of a second item I2 therein to generate a variability feature trend score vd1. The second trend extraction unit 512_3 may analyze features va2 to va4 of the first item I1 in the second long-term window group LWG2 to generate a variability feature trend score vc2 and may analyze features vb2 to vb4 of the second item I2 therein to generate a variability feature trend score vd2. As in the above description, the second trend extraction unit 512_3 may generate variability feature trend scores with respect to the remaining line groups window groups LWG3 to LWG7, and thus, additional description will be omitted to avoid redundancy.

The second trend extraction unit 512_3 may generate a short-term variability feature trend SKD2 based on the short-term feature window SWD to be similar to the way to generate the long-term variability feature trend LKD2 based on the long-term feature window LWD, and thus, additional description will be omitted to avoid redundancy.

As illustrated in FIG. 12, the ensemble prediction model 510 may analyze various trend features constituting time-series data in various ways. As the ensemble prediction model 510 processes the long-term time-series data LTD and the short-term time-series data STD independently of each other, it may be possible to comprehensively analyze the analysis results of long/short-term perspectives different in importance in determining time-series similarity. The ensemble prediction model 510 may distinguish a similarity difference according to a time-series trend difference that cannot be discriminated by a single trend feature through complex trends including a moving trend, a variability trend, a trend momentum trend, and the like.

FIG. 13 is a block diagram illustrating an example of a goodness-of-fit evaluation unit of FIG. 8. Referring to FIGS. 8 and 13, the goodness-of-fit evaluation unit 513 may include a long-term trend analysis unit 513_1, a short-term trend analysis unit 513_2, a goodness-of-fit calculation unit 513_3, first to fourth attention learning units CL1 to CL4, and first to fourth multiplexers MUX1 to MUX4.

The goodness-of-fit evaluation unit 513 may evaluate similarity of time-series data and goodness-of-fit (i.e., external prediction goodness-of-fit) of an external medical support system from a feature trend. The long-term trend analysis unit 513_1 may be implemented with a long short-term memory (LSTM) neural network. The short-term trend analysis unit 513_2 may be implemented with a long short-term memory (LSTM) neural network.

The goodness-of-fit evaluation unit 513 may receive feature trends extracted from the trend extraction unit 512, the health time-series data HTD, and the first and second external prediction results RTD1 and RTD2. The feature trends may include a feature trend for the health time-series data HTD, a feature trend for the first external prediction result RTD1, and a feature trend for the second external prediction result RTD2.

The feature trend for the health time-series data HTD may include a long-term feature trend LKD_HTD for health time-series data, and a short-term feature trend SKD_HTD for health time-series data. The feature trend for the first external prediction result RTD1 may include a long-term feature trend LKD_RTD1 for the first external prediction result RTD1 and a short-term feature trend SKD_RTD1 for the first external prediction result RTD1. The feature trend for the second external prediction result RTD2 may include a long-term feature trend LKD_RTD2 for the second external prediction result RTD2 and a short-term feature trend SKD_RTD2 for the second external prediction result RTD2.

The long-term feature trends LKD_HTD, LKD_RTD1, and LKD_RTD2 associated with the health time-series data HTD, the first external prediction result RTD1, and the second external prediction result RTD2 may include the long-term moving feature trend LKD1_1, the long-term trend transition feature trend LKD1_2, the long-term variability feature trend LKD2, the long-term slope feature trend LKD3_1, and the long-term variation feature trend LDK3_2.

The short-term feature trends SKD_HTD, SKD_RTD_1, and SKD_RTD2 associated with the health time-series data HTD, the first external prediction result RTD1, and the second external prediction result RTD2 may include the short-term moving feature trend SKD1_1, the short-term trend transition feature trend SKD1_2, the short-term variability feature trend SKD2, the short-term slope feature trend SKD3_1, and the short-term variation feature trend SDK3_2.

Long-term input data LID may include the health time-series data HTD, the first external prediction result RTD1, the second external prediction result RTD2, and the long-term feature trends LKD_HTD, LKD_RTD1, and LKD_RTD2 associated with the health time-series data HTD, the first external prediction result RTD1, and the second external prediction result RTD2.

Short-term input data SID may include the health time-series data HTD, the first external prediction result RTD1, the second external prediction result RTD2, and the short-term feature trends SKD_HTD, SKD_RTD1, and SKD_RTD2 associated with the health time-series data HTD, the first external prediction result RTD1, and the second external prediction result RTD2.

The first attention learning unit CL1 may receive the long-term input data LID. The first multiplexer MUX1 may receive an output of the first attention learning unit CL1 and the long-term input data LID and may output one of the output of the first attention learning unit CL1 and the long-term input data LID.

The second attention learning unit CL2 may receive the short-term input data SID. The second multiplexer MUX2 may receive an output of the second attention learning unit CL2 and the short-term input data SID and may output one of the output of the second attention learning unit CL2 and the short-term input data SID.

The long-term trend analysis unit 513_1 may receive an output of the first multiplexer MUX1. The long-term trend analysis unit 513_1 may output a long-term goodness-of-fit vector LV. The short-term trend analysis unit 513_2 may receive an output of the second multiplexer MUX2. The short-term trend analysis unit 513_2 may output a short-term goodness-of-fit vector SV.

The third attention learning unit CL3 may receive the long-term goodness-of-fit vector LV. The third multiplexer MUX3 may receive the long-term goodness-of-fit vector LV and an output of the third attention learning unit CL3 and may output one of the long-term goodness-of-fit vector LV and the output of the third attention learning unit CL3.

The fourth attention learning unit CL4 may receive the short-term goodness-of-fit vector SV. The fourth multiplexer MUX4 may receive the short-term goodness-of-fit vector SV and an output of the fourth attention learning unit CL4 and may output one of the short-term goodness-of-fit vector SV and the output of the fourth attention learning unit CL4.

The goodness-of-fit calculation unit 513_3 may receive an output of the third multiplexer MUX3 and an output of the fourth multiplexer MUX4. The goodness-of-fit calculation unit 513_3 may calculate and output prediction goodness-of-fit (i.e., external prediction goodness-of-fit) of an external medical support system.

The long-term goodness-of-fit vector LV may include information for determining whether external prediction results fit as an ensemble prediction result in a long-term trend. The short-term goodness-of-fit vector SV may include information for determining whether external prediction results fit as an ensemble prediction result in a short-term trend.

In an embodiment, the health time-series data HTD, the first external prediction result RTD1, the second external prediction result RTD2, and the long-term feature trends LKD_HTD, LKD_RTD1, and LKD_RTD2 corresponding to each of a plurality time points may be sequentially input to the LSTM neural network of the long-term trend analysis unit 513_1. As a result, a feature vector of a previous time may be applied to generate a feature vector of a next time, and the long-term trend analysis unit 513_1 may calculate a long-term trend goodness-of-fit feature vector (i.e., the long-term goodness-of-fit vector LV) of the external prediction results at a prediction time point.

In an embodiment, the health time-series data HTD, the first external prediction result RTD1, the second external prediction result RTD2, and the short-term feature trends SKD_HTD, SKD_RTD1, and SKD_RTD2 corresponding to each of a plurality time points may be sequentially input to the LSTM neural network of the short-term trend analysis unit 513_2. As a result, a feature vector of a previous time may be applied to generate a feature vector of a next time, and the short-term trend analysis unit 513_2 may calculate a short-term trend goodness-of-fit feature vector (i.e., the short-term goodness-of-fit vector SV) of the external prediction results at the prediction time point.

The first attention learning unit CL1 may learn the attention with respect to long-term feature trends LKD. For example, the attention means learning the degree of contribution to a learning result with respect to each input and weighting an input such that the attention made to an input with the great degree of contribution.

The first to fourth attention learning units CL1 to CL4 F may receive I₁,I₂ . . . Iₙ and may return A like Equation 8 below.

A=F(S₁,S₂ . . . Sₙ) [Equation 8]

In this case, a result may be A={a₁, a₂ . . . aₙ}, an arbitrary aₖ means a weight for an arbitrary input (0≤aₖ≤1, Σ₁ⁿaₖ=1). As such, an input feature A·1={(a₁*I₁, a₂*I₂ . . . aₙ*Iₙ}) to which a weight is applied may be input to the long-term trend analysis unit 513_1 or the short-term trend analysis unit 513_2. For example, the weighted long-term input data may be input to the long-term trend analysis unit 513_1, and the weighted short-term input data may be input to the short-term trend analysis unit 513_2.

In an embodiment, the second attention learning unit CL2 may learn the attention with respect to short-term feature trends SKD. The third attention learning unit CL3 may learn the attention with respect to the long-term goodness-of-fit vector LV. The fourth attention learning unit CL4 may learn the attention with respect to the short-term goodness-of-fit vector SV. The first to fourth attention learning units CL1 to CL4 may be implemented with a fully connected layer of an artificial neural network.

The goodness-of-fit calculation unit 513_3 may receive the long-term goodness-of-fit vector LV and the short-term goodness-of-fit vector SV and may calculate the prediction goodness-of-fit (or external prediction goodness-of-fit) of the external medical support system. That is, the goodness-of-fit calculation unit 513_3 may calculate a weight indicating whether to fit, as an ensemble prediction result of an external prediction result.

The external prediction goodness-of-fit may be calculated by Equation 9 corresponding to an external predictive value vector <pA, . . . , pN>.

$$External prediction goodness - of - fit = < sA , … , sN > vector in which {\sum }_{1}^{n} = 1 \qquad [ Equation 9 ]$$

In an embodiment, the goodness-of-fit calculation unit 513_3 may be implemented with a fully connected layer of an artificial neural network.

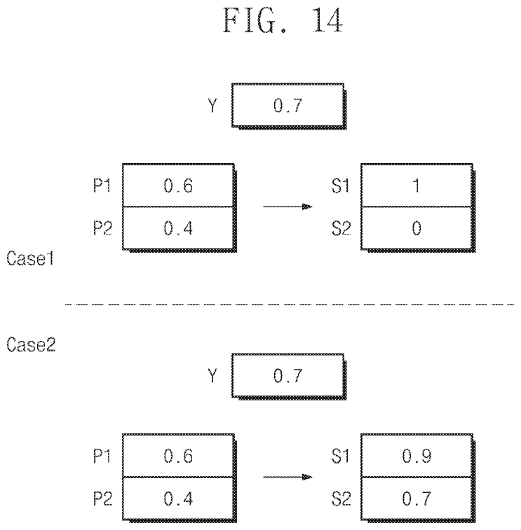

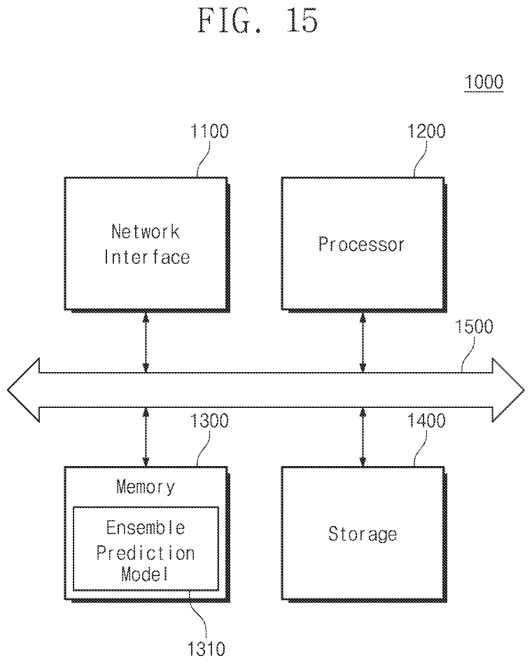

FIG. 14 is a diagram illustrating an operation of an error learning unit of FIG. 8. Referring to FIGS. 8 and 15, the error learning unit 514 may receive the external prediction goodness-of-fit from the goodness-of-fit evaluation unit 513. The error learning unit 514 may generate real external goodness-of-fit based on predictive values of the prediction time and an experimental value of the prediction time. The error learning unit 514 may calculate an error based on the received external prediction goodness-of-fit and the real external goodness-of-fit. The error learning unit 514 may adjust a parameter group for an operation of each component of the ensemble prediction model 510 such that an error is minimized.

The real external goodness-of-fit may include a first real external goodness-of-fit S1 corresponding to the first external prediction result RTD1 and a second real external goodness-of-fit S2 corresponding to the second external prediction result RTD2. For example, the first real external goodness-of-fit S1 may indicate real external goodness-of-fit of the second medical support system 12, and the second real external goodness-of-fit S2 may be real external goodness-of-fit of the third medical support system 13.

In an embodiment, the error learning unit 514 may generate real external goodness-of-fit “S” for error learning. The error learning unit 514 may calculate the real external goodness-of-fit “S” based on a first predictive value P1 of the prediction time of the first external prediction result RTD1, a second predictive value P2 of the prediction time of the second external prediction result RTD2, and an experimental value “Y” of the prediction time.

For example, the error learning unit 514 may calculate a first difference between the first external prediction result RTD1 (i.e., the first predictive value P1) corresponding to the prediction time and the experimental value “Y” of the prediction time. The error learning unit 514 may calculate a second difference between the second external prediction result RTD2 (i.e., the second predictive value P2) corresponding to the prediction time and the experimental value “Y” of the prediction time. When the first difference is greater than the second difference, the first real external goodness-of-fit S1 may be set to “0”, and the second real external goodness-of-fit S2 may be set to “1”. When the first difference is smaller than the second difference, the first real external goodness-of-fit S1 may be set to “1”, and the second real external goodness-of-fit S2 may be set to “0”. That is, real external goodness-of-fit of an external medical support system corresponding to a difference being the smallest from among a plurality of differences may be set to “1”, and real external goodness-of-fit of each of the remaining external medical support systems may be set to “0”.

For example, it is assumed that the first experimental value P1 is “0.6”, the second experimental value P2 is “0.4”, and the experimental value “Y” is “0.7”. In this case, the first difference may be “0.1”, and the second difference may be “0.3”. Because the first difference of “0.1” is smaller than the second difference of “0.3”, the first real external goodness-of-fit S1 may be set to “1”, and the second real external goodness-of-fit S2 may be set to “0”.

In an embodiment, the error learning unit 514 may determine a result of subtracting a difference between the experimental value “Y” and a predictive value from a maximum error (e.g., “1”), as the real external goodness-of-fit. For example, it is assumed that the first experimental value P1 is “0.6”, the second experimental value P2 is “0.4”, and the experimental value “Y” is “0.7”. In this case, the first difference may be “0.1”, and the second difference may be “0.3”. The first real external goodness-of-fit S1 may be “0.9”, and the second real external goodness-of-fit S2 may be “0.7”.

FIG. 15 is a block diagram illustrating an example of a prediction system of FIG. 1. Referring to FIG. 15, an prediction system 1000 may include a network interface 1100, a processor 1200, a memory 1300, storage 1400, and a bus 1500. For example, the prediction system 1000 may be implemented with a server, but is not limited thereto. It is assumed that the prediction system 1000 is the first prediction system PS of the first medical support system 11.

The network interface 1100 may be configured to communicate with the external medical support systems 12 to 14 over the network NT of FIG. 1. The network interface 1100 may provide data received over the network NT to the processor 1200, the memory 1300, or the storage 1400 over the bus 1500. The network interface 1100 may output partial time-series data to the external medical support systems 12 to 14 together with the prediction request of the processor 1200. Also, the network interface 1100 may receive external prediction results that are generated in response to the prediction result request and the partial time-series data.

The processor 1200 may function as a central processing unit of the prediction system 1000. The processor 1200 may perform a control operation and a computation/calculation operation that are required for data management, learning, and prediction of the prediction system 1000. For example, under control of the processor 1200, the network interface 1100 may send the partial time-series data to the external medical support systems 12 to 14 and may receive external prediction results from the external medical support systems 12 to 14. Under control of the processor 1200, an ensemble result may be calculated by using the ensemble prediction model. The processor 1200 may operate by utilizing a computation/calculation space of the memory 1300 and may read files for driving an operating system and execution files of applications from the storage 1400. The processor 1200 may execute the operating system and the applications.

The memory 1300 may store data and program codes that are processed by the processor 1200 or are scheduled to be processed by the processor 1220. For example, the memory 1300 may store external prediction results, pieces of information for managing the external prediction results, pieces of information for calculating an ensemble result, and pieces of information for building a prediction model. The memory 1330 may be used as a main memory of the prediction system 1000. The memory 1330 may include a dynamic random access memory (DRAM), a static RAM (SRAM), a phase-change RAM (PRAM), a magnetic RAM (MRAM), a ferroelectric RAM (FeRAM), a resistive RAM (RRAM), or the like.

An ensemble prediction model 1310 may be loaded and executed onto the memory 1300. The ensemble prediction model 1310 corresponds to the ensemble prediction model 510 of FIG. 2. The ensemble prediction model 1310 may be a portion of a calculation space of the memory 1300. In this case, the ensemble prediction model 1310 may be implemented by firmware or software. For example, the firmware may be stored in the storage 1400 and may be loaded onto the memory 1300 upon executing the firmware. The processor 1200 may execute the firmware loaded onto the memory 1300.

The storage 1400 may store data generated for the purpose of long-time storage by the operating system or the applications, files for driving the operating system, execution files of the applications, etc. For example, the storage 1400 may store files for execution of the ensemble prediction model 1310. The storage 1400 may be used as an auxiliary storage device of the prediction system 1000. The storage 1400 may include a flash memory, a PRAM, an MRAM, a FeRAM, an RRAM, etc.

The bus 1500 may provide a communication path between the components of the prediction system 1000. The network interface 1100, the processor 1200, the memory 1300, and the storage 1400 may exchange data with each other over the bus 1500. The bus 1500 may be configured to support various communication formats used in the prediction system 1000.

According to an embodiment of the present disclosure, a more accurate ensemble prediction result may be provided by extracting multiple trend features from health time-series data of a patient and a prediction result of an external medical support system and utilizing the trend features in analyzing similarity between the patient's health time-series data and the prediction result.

While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. An operation method of a health state prediction system which includes an ensemble prediction model, the method comprising:
sending a prediction result request for health time-series data to a first external medical support system and a second external medical support system;
receiving a first external prediction result associated with the health time-series data from the first external medical support system;
receiving a second external prediction result associated with the health time-series data from the second external medical support system;
generating long-term time-series data and short-term time-series data for each of the health time-series data, the first external prediction result, and the second external prediction result;
extracting a first long-term trend and a second long-term trend based on the long-term time-series data;
extracting a first short-term trend and a second short-term trend based on the short-term time-series data;
calculating external prediction goodness-of-fit based on the first and second long-term trends and the first and second short-term trends; and
generating an ensemble prediction result based on the external prediction goodness-of-fit and the first and second external prediction results.

2. The method of claim 1, further comprising:
calculating an error based on the calculated external prediction goodness-of-fit and a real external goodness-of-fit; and
adjusting a parameter of the ensemble prediction model based on the error.

3. The method of claim 2, wherein the real external goodness-of-fit is generated based on an experimental value of a prediction time point, a first external experimental value corresponding to the prediction time point, and a second external prediction result corresponding to the prediction time point.

4. The method of claim 1, wherein the number of features included in the long-term time-series data is equal to the number of features included in the health time-series data, and
wherein the number of features included in the short-term time-series data is less than the number of features included in the health time-series data.

5. The method of claim 1, wherein the first and second short-term trends and the first and second long-term trends correspond to at least one of a moving trend feature, a variability trend feature, or a moving momentum trend feature.

6. The method of claim 5, wherein the moving trend feature includes a moving feature and a trend transition feature, and the moving momentum trend feature includes a slope feature and a variation feature.

7. The method of claim 5, wherein the moving trend feature indicates a gradual change trend of a value of the long-term time-series data or the short-term time-series data,
wherein the variability trend feature includes a magnitude, a pattern, and a period of variability of a value in the long-term time-series data or the short-term time-series data, and
wherein the moving momentum trend feature indicates a change direction including an increase and a decrease of the long-term time-series data or the short-term time-series data, and a strength for the change direction.

8. The method of claim 1, wherein the extracting of the first and second long-term trends based on the long-term time-series data includes:
extracting features belonging to a window time interval from the long-term time-series data to generate a long-term feature window;
generating the first long-term trend based on the long-term feature window; and
generating the second long-term trend based on the long-term feature window.

9. The method of claim 1, wherein the calculating of the external prediction goodness-of-fit based on the first and second long-term trends and the first and second short-term trends includes:
generating a long-term goodness-of-fit vector based on the first and second long-term trends;
generating a short-term goodness-of-fit vector based on the first and second short-term trends; and
calculating the external prediction goodness-of-fit based on the long-term goodness-of-fit vector and the short-term goodness-of-fit vector.

10. The method of claim 9, wherein the generating of the long-term goodness-of-fit vector based on the first and second long-term trends includes:
generating a first long-term goodness-of-fit feature vector based on the health time-series data corresponding to a first time point, the first and second external prediction results corresponding to the first time point, and the first and second long-term trends corresponding to the first time point; and
generating a second long-term goodness-of-fit feature vector based on the first long-term goodness-of-fit feature vector, the health time-series data corresponding to a second time point after the first time point, the first and second external prediction results corresponding to the second time point, and the first and second long-term trends corresponding to the second time point.

11. A health state prediction system comprising:
a first medical support system including a first clinical decision support system and a first prediction system;
a second medical support system including a second clinical decision support system and a second prediction system; and
a third medical support system including a third clinical decision support system and a third prediction system,
wherein the first prediction system includes:
a predictor management device connected with the first clinical decision support system, and configured to receive an ensemble prediction request and health time-series data from the first clinical decision support system and to send an ensemble prediction request to the first clinical decision support system;
an ensemble prediction device configured to receive a prediction execution request from the predictor management device, to send an external prediction result request to a predictor interworking device in response to the prediction execution request, to receive merged data from the predictor interworking device, to input the merged data to an ensemble prediction model, and to receive the ensemble prediction result from the ensemble prediction model, wherein the predictor interworking device is configured to:
send a prediction result request and the health time-series data to the second and third medical support systems in response to the external prediction result request;
receive a first external prediction result from the second medical support system;
receive a second external prediction result from the third medical support system;
merge the health time-series data and the first and second external prediction results to generate the merged data; and
an ensemble prediction model configured to receive the merged data from the ensemble prediction device and to generate the ensemble prediction result.

12. The health state prediction system of claim 11, further comprising:
a time-series prediction device configured to receive a prediction execution request and the health time-series data from the predictor interworking device, to input the health time-series data to a time-series prediction model, and to receive the time-series prediction result from the time-series prediction model.

13. The health state prediction system of claim 12, wherein the predictor interworking device is configured to merge the health time-series data, the first and second external prediction results, and the time-series prediction result to generate the merged data.
